(19) United States
(12) Patent Application Publication
(10) Pub. No.: US 2005/0107846 A1
(43) Pub. Date: May 19, 2005

(54) Implantable medical drive with text messaging capability

(75) Inventor: Robert J. Sweeney, Woodbury, MN (US)

Correspondence Address: NIKOLAI & MERSEREAU, P.A., 900 SECOND AVENUE SOUTH, SUITE 820, MINNEAPOLIS, MN, 55402, US

(73) Assignee: Cardiac Pacemakers, Inc., St. Paul, MN (US)

(21) Appl. No.: 10/713,813

(22) Filed: Nov. 14, 2003

(51) Int. Cl. A61N 1/362

(52) U.S. Cl. 607/60; 607/32

(57) ABSTRACT

A method of communicating text messages to a clinician as a reminder that certain program changes are to be made to a medical tissue stimulating device implanted in a patient. An external programmer capable of transmitting and receiving radio-frequency messages to and from the implant and visually displaying information is used to transmit text messages along with a text message initiation date and a text message termination date to the implanted device. These data are stored in a memory of the implanted device's microprocessor-based controller. Subsequently, at the time of a regularly scheduled patient follow-up, the implant may be interrogated by the external programmer and the text message read out and displayed if the time of the interrogation falls between the text message initiation date and its termination date. The method may further involve assigning a priority flag to the text message when it is stored in the implanted device so that upon interrogation, the text message may be displayed in a manner dictated by the priority code.

### BACKGROUND OF THE INVENTION

I. Field of the Invention

This invention relates generally to implantable medical devices having a telemetry capability for transmitting information to and receiving information from an external programmer/monitor, and more particularly to an implantable device capable of storing text messages and relaying those messages to an external programmer/monitor for informing a clinician of certain events so that timely action can be taken.

II. Discussion of the Prior Art

Since the mid-70's implantable medical devices, such as implantable pacemakers, have been programmable from a location outside of the body. The Fischer U.S. Pat. No. 4,304,238 assigned to the assignee of the present invention was one of the first to disclose a digital, programmable, implantable pacemaker that would allow a clinician, having an external programmer, to change the pacing parameters of the implanted device. Thus, pacing rate, stimulation pulse width, stimulation pulse amplitude could be changed to meet a patient's need by transmitting digital values from an external programmer to a data storage register or memory in the implanted device. As the technology progressed, a myriad of additional parameters and operational modes of pacemakers and automatic implantable cardiac defibrillators can now be reprogrammed following implant.

Present-day implantable devices allow for bi-directional communication so that not only can data be transmitted to the implanted device, but also the implanted device may transmit information on its operational status to the external programmer. In this fashion, physiologic data, including electrograms can be temporarily stored in the memory of an implanted device and subsequently, upon interrogation by the external programmer, the information is transmitted to the programmer for display and interpretation by a clinician.

One great difficulty in performing a clinical trial is the tracking of patients that have been enrolled as participants in the study or trial. As an example, a patient with an implantable pacemaker or pacemaker/defibrillator may be enrolled in a particular study involving a protocol where certain programmable parameters of the implantable device are to be changed at different preestablished times and a determination is then to be made whether such change improved or worsened cardiac performance of that patient. While the relevant data for insuring that the protocol is followed may be recorded on the patient's medical chart, it would be convenient and beneficial if a reminder message could be presented to the clinician on the display screen of the programmer/monitor when the implanted device is being interrogated by the programmer. The method of the present invention provides such a system.

### II. SUMMARY OF THE INVENTION

The present invention is directed to a method of communicating text messages between an implantable medical device and an external programmer where the external programmer has the capability of transmitting and receiving telemetry signals and for visually displaying information to a person. The external programmer is used in combination with an implantable medical device having a microprocessor-based controller with a memory for not only storing a program of instructions and operands executable by the microprocessor-based controller, but also text messages of predetermined number and length. The implantable device also includes a telemetry link that allows duplex communication with the external programmer. Initially, the external programmer is used to enter text messages, along with a text message initiation date and a text message termination date. Subsequently, at the time of an office visit by the patient, the memory of the implantable device is interrogated using the external programmer and the text message is read out over the telemetry link too the external programmer for visual display when the time of the interrogation falls between the previously stored initiation date and the termination date.

It is a further feature of the present invention that the text message be assigned a priority flag when it is transmitted to the implantable medical device. Subsequently, when the text message is read out, its priority flag can be used to determine the frequency at which the text message read out from the memory of the implantable device is displayed on the external programmer. The priority flag may also determine the manner in which the text message is displayed.

### DESCRIPTION OF THE DRAWINGS

The foregoing features, objects and advantages of the invention will become apparent to those skilled in the art from the following detailed description of a preferred embodiment, especially when considered in conjunction with the accompanying drawings in which like numerals in the several views refer to corresponding parts.

FIG. 1 is a system diagram of the hardware in which the algorithm of the present invention is carried out;

FIG. 2 is a block diagram representation of an implantable medical device used in carrying out the method of the present invention;

FIGS. 3a-3c is a flow chart of the algorithm by which text messaging is implemented;

FIG. 4 is an illustrative screen print of a implantable medical device's external programmer/monitor display screen incorporating a “message waiting” indicator; and

FIG. 5 shows the screen print of FIG. 4 with a text message readout from the implanted device superimposed thereon.

### DESCRIPTION OF THE PREFERRED EMBODIMENT

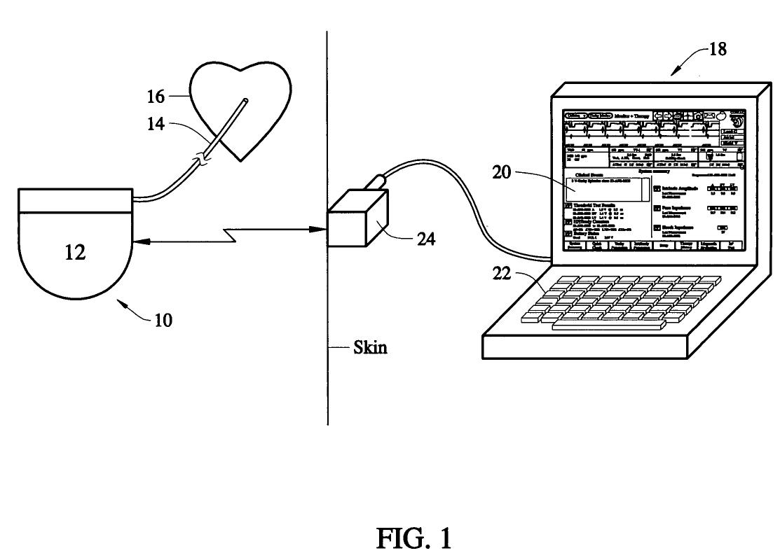

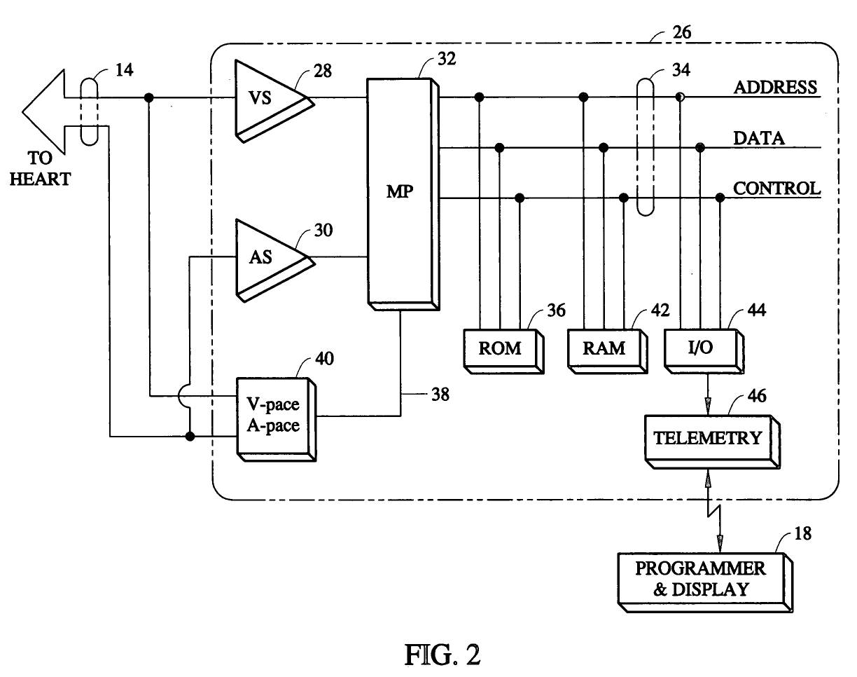

Referring to FIG. 1, there is shown an implantable medical device, such as a pacemaker, an implantable automatic cardiac defibrillator or a neural stimulator. It is indicated generally by numeral 10 and includes the electronic circuitry of FIG. 2 housed in a hermetically sealed can 12. A pacing lead, as at 14, couples the implantable device to target tissue, here shown as the heart 16. Located within a clinician's office is a programmer/monitor 18 having a visual display screen 20 and a data entry keyboard 22. The external programmer 18 may be placed in communication with the implantable device by way of a RF telemetry link also contained within the can or housing 12. A wand transducer 22 is connected to the programmer/monitor and functions as a transmitting/receiving antenna whereby programmable parameters may be read into a memory module so that operating features of the device can be changed without having to explant the device 10.

In accordance with the present invention, not only can programmable parameters be transmitted to the implantable device through the skin, but also text messages, composed on the keyboard 22 or otherwise made available to the programmer 18 may be transmitted to and stored in the memory of the implantable device 10.

Turning to FIG. 2, there is shown a block diagram of a typical implantable pacemaker. The housing 12 is represented by the broken line box 26. Contained within it is a ventricular sense amplifier 28, an atrial sense amplifier 30, each of which has its output connected to a microprocessor-based controller module 32. Connected to the microprocessor-based controller 32 by means of a bus structure 34 is a read-only memory (ROM) 36. This memory typically will store the software program executable by the microprocessor within the microprocessor-based controller 32. It then delivers control signals over line 38 to a pulse generator 40 whereby the pulse generator is made to deliver ventricular stimulating pulses and atrial stimulating pulses over the medical lead 14 to the heart at predetermined times defined by the program stored in the ROM 36.

A random access memory (RAM) 42 is also connected by the bus 34 to the microprocessor-based controller 32 and is typically used to store programmable parameters and operands utilized by the program stored in the ROM 36. Programmable parameters may include stimulating pulse amplitude, stimulating pulse width, as well as many, many other such parameters commonly found in present-day pacemakers and defibrillators. In accordance with the present invention, there is also stored in the RAM 42 multi-character text messages. Associated with each of the text messages is an initiation date, a termination date and a priority flag or code.

With continued reference to FIG. 2, the implantable device also will include an input/output controller 44, which is coupled to a RF transceiver comprising the telemetry link 46 whereby data can be transmitted in a duplex mode between the implantable device 10 and the external programmer 18. Without limitation, the telemetry link may be configured like that disclosed in the Von Arx et al. U.S. Pat. No. 6,574,510, also assigned to applicant's assignee, the contents of which are hereby incorporated by reference.

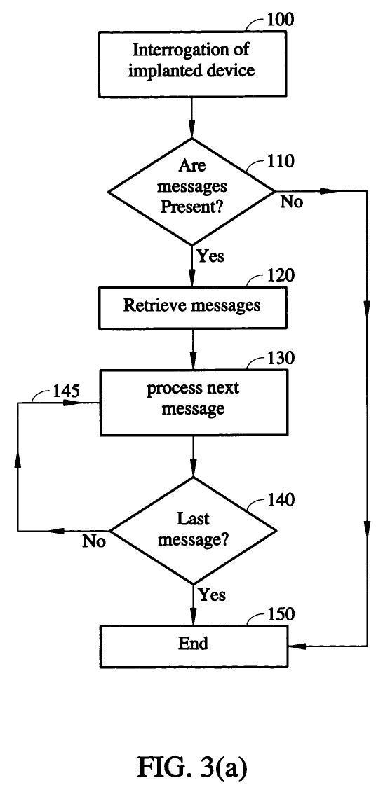

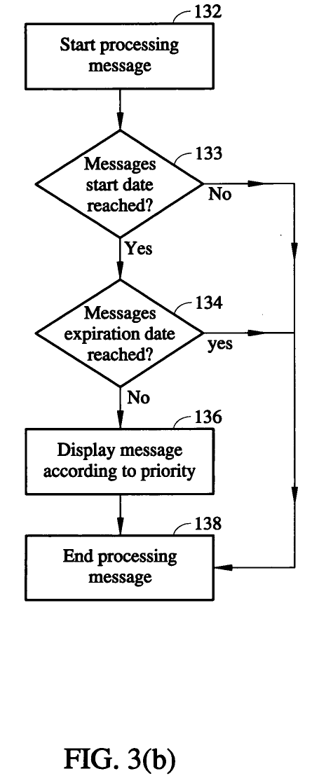

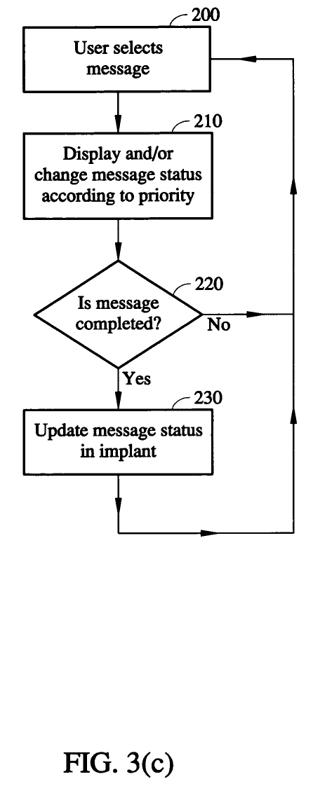

FIG. 3a through 3c comprise software flow diagrams of the algorithms employed in retrieving stored text messages from an implanted device.

Referring to FIG. 3(a), at operation block 100, the implanted device is interrogated by an external programmer or another interrogating device. As part of the interrogation, it is determined at decision block 110 if messages are stored in the implanted device and, if so, at operation block 120 the messages are retrieved over the telemetry link. The retrieved messages are processed at operation block 130 and following such processing a test is made at decision block 140 to determine if any further messages require processing. If the current message is not the last message, control loops back over line 145 and the next message is processed. If the test at decision block 140 reveals that it is the last message in a queue, message retrieval and display ends at 150.

The operations carried out in block 130 are reflected in the flowchart of FIG. 3(b). At operation block 132 the processing of an individual message begins. At decision block 133, it is determined whether the current date has reached the start date of the message processed. If not, the processing for the current message ends at block 138. If the current date has reached the message start date then a test is made at decision block 134 to determine if the current date has exceeded the expiration date for the message. If the message expiration date has come and gone, i.e., it has expired, then processing for the current message ends at block 138. However, if the current message has not yet expired, then, at operation block 136, the message is displayed on the programmer screen or other interrogating device according to the priority assigned to the message.

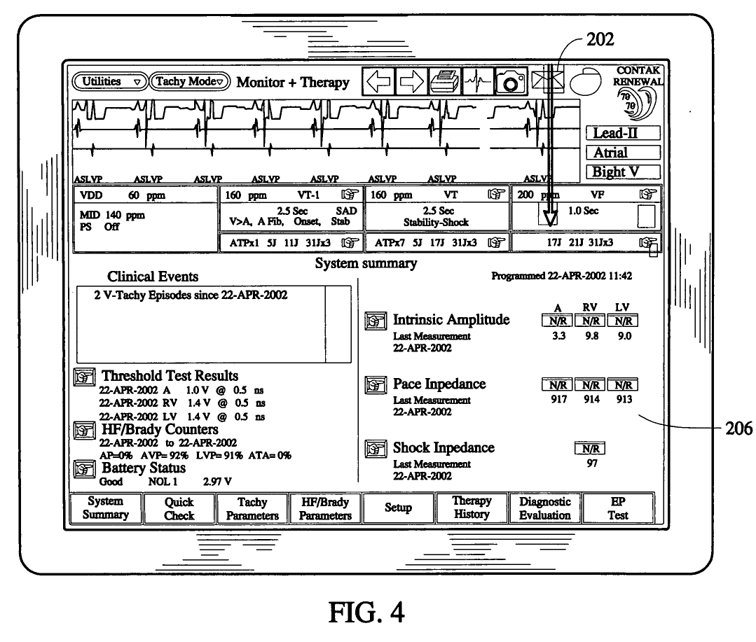

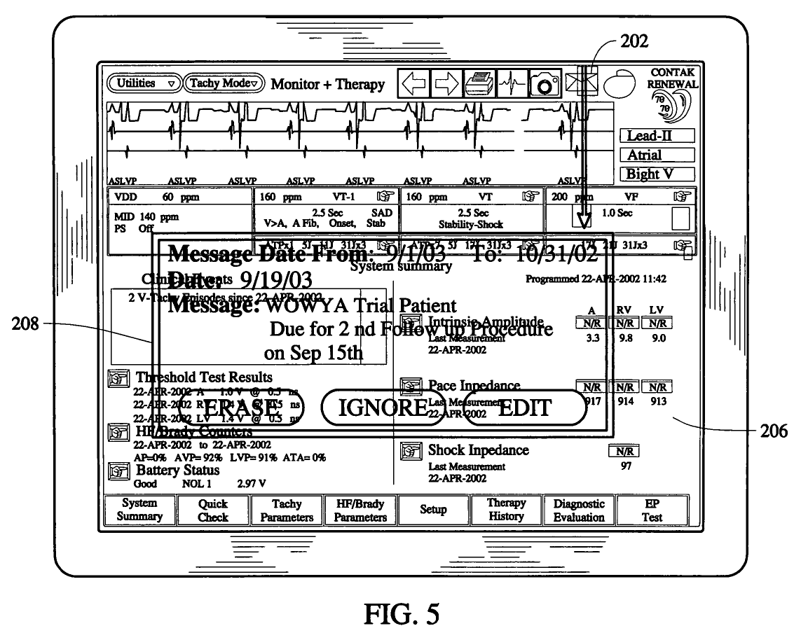

FIG. 3(c) is a flowchart of the algorithm for user response and disposition of the displayed messages. At operation block 200, the clinician selects a message, either by clicking on a “message waiting” icon 202 (FIG. 4) appearing on the display screen 206 of the programmer, selecting a message from a list of messages in a drop-down menu; or directly responding to a message already displayed on the screen. By programming the “message waiting” icon 202 to blink or to be presented in a predetermined color, the priority status of the message can be indicated. The priority status may then reflect the frequency that the text message is displayed on the monitor screen. At operation block 210, the message contents of the selected message are displayed to the clinician. FIG. 5 shows a typical text message 208 read out from the implanted medical device and overlayed on the programmer/monitor 18 display screen 206. A test is then made at decision block 220 to determine if the selected message has been acted upon. If the action called for by the message has not been carried out, control loops back to block 200 and the message remains in the queue until appropriate action has been taken, at which point the message can be erased, have its priority changed, have its action date changed or some other response to the message is effected. If, however, the selected message is complete, then the status for the selected message is updated in the implanted device. See operation block 230. By positioning a cursor on one of the “ERASE”, “IGNORE” or “EDIT” areas and clicking a mouse, the text message originating in the implanted medical device can be deleted from the screen 206, the message can be acted on at a later time or its priority and/or dates can be altered, respectively. The next message may then be selected at block 200.

It can thus be seen that the present invention provides a method of communicating text messages between an implantable medical device and an external programmer, where the message can be visually displayed. Although the present method has been described with reference to specific details of certain embodiments, it is not intended that such details should be regarded as limitations upon the scope of the invention except as and to the extent that they are included in the accompanying claims.

1. A method of communicating text messages between an implantable medical device and an external programmer, comprising the steps of
(a) providing an external programmer for transmitting and receiving data signals and for visually displaying information to a person;
(b) providing an implantable medical device having a microprocessor-based controller with a memory for storing text messages and a telemetry link for allowing duplex communication with said external programmer;
(c) transmitting at least one text message from the external programmer to the implantable medical device over the telemetry link along with a text message initiation date and a text message termination date;
(d) storing the at least one text message in said memory;
(e) subsequently periodically interrogating the memory with said external programmer and reading out the at least one text messages over the telemetry link to the external programmer for visual display when the time of said interrogation falls between the initiation date and the termination date.

2. The method as in claim 1 and further including the step of
(a) assigning a priority flag to the text message transmitted to the implantable medical device.

3. The method as in claim 2 wherein said priority flag determines the manner in which the at least one text messages is displayed.

4. The method as in claim 3 wherein said priority flag determines the frequency at which the at least one text message read out from said memory is displayed on the external programmer.

5. The method as in claim 2 and further including the step of changing the priority flag when the at least one text message is read out to the external programmer.

6. The method as in claim 1 and further including the step of updating the status of a given text message in the implantable medical device following the performance of a responsive action by a clinician to said text message presented on the visual display.

7. The method as in claim 6 wherein the status updating comprises erasing the text message from the memory of the implantable medical device.

8. The method as in claim 6 wherein the status updating comprises revising a priority flag assigned to the message.

9. The method as in claim 6 wherein the status updating comprises changing one of said text message initiation date and text message termination date.
